(19) United States
(12) Patent Application Publication
(10) Pub. No.: US 2024/0034974 A1
(43) Pub. Date: Feb. 1, 2024

(54) CULTURE DEVICE FOR BIOCHEMICAL TESTING

(71) Applicant: National Tsing Hua University, Hsinchu City (TW)

(72) Inventors: Chi-Shuo Chen, Hsinchu City (TW); Jui-Hsun Chang, Hsinchu City (TW); Lin-Yen Yu, Hsinchu City (TW)

(21) Appl. No.: 18/095,547

(22) Filed: Jan. 11, 2023

(30) Foreign Application Priority Data
Jul. 26, 2022 (TW) 111127951

(51) Int. Cl. C12M 3/00; C12M 1/00; B01L 3/00

(52) U.S. Cl. CPC C12M 23/42; C12M 41/14; B01L 3/5027; B01L 2300/0861; B01L 2300/0816

(57) ABSTRACT

The present invention provides a culture device for biochemical testing. The culture device includes a first plate and a second plate, and the second plate is opposite to the first plate. The first plate and the second plate form an incubation cassette and a supply channel, and the incubation cassette is connected to the supply channel by a microstructure. The present invention can provide side views of multi-layered cellular samples, such as corneal tissue, and can be easily combined with other techniques for more comprehensive detection. In addition, applicable industries or products include: cell culture devices, in vitro clinical specimen culture, 3D cell culture, high-throughput drug screening, tumor invasion assessment, cell biology research, personalized precision medicine, etc.

### CROSS REFERENCE TO RELATED APPLICATIONS

This present application claims the benefits of Taiwan Patent Application No. 111127951, filed on Jul. 26, 2022.

### FIELD OF THE INVENTION

The present invention relates to the design of a culture device for biochemical testing. More particularly, the present invention relates to an incubation cassette both for cell culturing and biochemical testing under 3-dimensional environment.

### BACKGROUND OF THE INVENTION

According to data from Stratistics MRC, a well-known market analysis company, the global 3D cell culture market was US$1,032.04 million in 2020 and is expected to reach US$3,801.1 million by 2028, growing at a CAGR of 17.7% during the forecast period. Growing focus on developing alternatives to animal testing and rising incidence of chronic diseases, and the market growth are also driven. However, the high cost of cell biology research is restraining the market growth.

3D cell culture is the practice of augmenting biological cells to interact with their surroundings in all three dimensions, and that allows cells to develop in their natural environment under in vivo conditions. Cell-based assays are widely used in personalized precision medicine because they can quickly and efficiently evaluate the response of target cells under various experimental conditions. Compared with traditional 2D cell culture, 3D cell culture technology has been developed to simulate the growth environment of in vivo tissues. The scaffold-free 3D cell culture technology is suitable for densely interacting lamellar tissues, such as skin, cornea, or tumor tissue. However, most of the current research still relies on the routine analysis of 2D cell culture. Rather than the 2D cell culture model, the 3D culture system has several advantages, such as more accurate cell influence on cell culture microenvironment, oxygen and nutrient gradients, interactions between cells and their cells increase the extracellular medium (ECM), more accurately characterizing cell proliferation. Many different 3D cell culture technologies and platforms thereof have been proposed today, but there are still several problems with the existing methods.

There are four major problems that cannot be overcome as following:

1. The traditional cell culture environment can only obtain the information of the top view, and it is difficult to directly observe the structure and its changes of the depth (z-axis); even the current technology still needs to cooperate with freezing and cutting sections of paraffin-embedded tissues to observe the depth (z-axis) of cell structure.

2. Microfluidic culture systems often require additional special equipment (pumps, hoses, plug-ins, etc.), and are not easy to operate. Layer-by-layer culture requires special materials as scaffolds, and then stacks the layer-by-layer scaffolds into a 3D structure. The operation is complicated.

3. Since the 3D cultured tissue has no blood vessels to transport nutrients, the nutrients and wastes required for its internal metabolism can only be exchanged by diffusion, which also limits its three-dimensional thickness (z-axis).

4. Limited by the number of starting cells required: clinical samples are not easy to obtain, the number is scarce, and most of them are cultured in cell-culture dishes with small area size (such as 384 or 96 wells).

Although there are different methods for 3D culture in the current culture technology and its devices, such as the permeable cell culture dishes “Transwell”, it needs to be embedded and sliced to observe the depth (z-axis) structure to obtain a side view. However, its original side-view may be destroyed during the slicing process. Even with a specially designed microfluidic system, instant depth (z-axis) observation can be performed, the operation is often inconvenient and requires a high cell number. Also, the traditional small-sized cell culture dishes can only be loaded with limited volume of the nutrient solution, so the nutrient solution often needs to be replaced frequently. The cell samples are easily taken away with the nutrient solution during replacement, to result the loss of cell samples.

Currently known technical devices mostly focus on how to cultivate 3D layered multicellular structures, but do not solve or provide a device that can easily be observed and obtained side views of multi-layered cellular structures during experiments or testing. To sum up the above, there is no cell culture device on the market that can simultaneously meet the aforementioned experimental or detection requirements.

### SUMMARY OF THE PRESENT INVENTION

The present invention provides a culture device for biochemical testing, and can provide side views of multi-layered cellular samples, such as corneal tissue, and can be easily combined with other techniques for more comprehensive detection. In addition, applicable industries or products include: cell culture devices, in vitro clinical specimen culture, 3D cell culture, high-throughput drug screening, tumor invasion assessment, cell biology research, personalized precision medicine, etc.

In order to achieve one or more of the above objections, one embodiment of the present invention provides a culture device for biochemical testing (or called as a cell incubation cassette). The culture device includes a first plate and a second plate, the second plate is opposite to the first plate, and the first plate and the second plate form an incubation cassette and a supply channel, wherein the incubation cassette is connected to the supply channel by a micro structure.

### BRIEF DESCRIPTION OF THE DRAWINGS

FIG. 1, FIG. 2A and FIG. 2B illustrate, respectively, a three-dimensional view, a side view and a top view of the culture device for biochemical testing, according to one embodiment of the present invention.

FIG. 3 illustrates a front view and a partial enlarged schematic of the culture device 100 for biochemical testing, according to one embodiment of the present invention.

FIG. 4A, FIG. 4B and FIG. 4C show, respectively, a front view, a top view and a side vide of the physical culture device 100 for biochemical testing, according to one embodiment of the present invention.

FIG. 5 and FIG. 6 show, respectively, a side image (with 5000 μm as the standard scale) and a partial image thereof (with 1000 μm as the standard scale) of the co-culture of lamellar cells in the culture device.

### DETAILED DESCRIPTION OF THE INVENTION

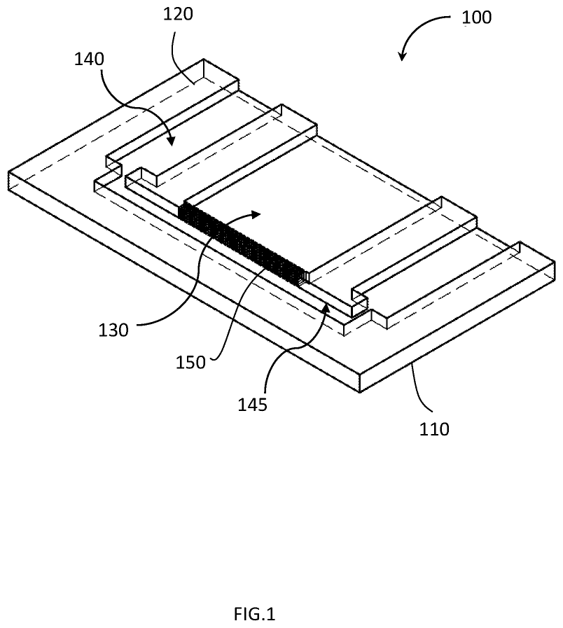

The present invention provides a culture device for biochemical testing in order to provide side views of multi-layered cellular samples, such as corneal tissue, and can be easily combined with other techniques for more comprehensive detection. Refer to FIG. 1, FIG. 2A and FIG. 2B, illustrate, respectively, a three-dimensional view, a side view and a top view of the culture device for biochemical testing, according to one embodiment of the present invention. The culture device 100 for biochemical testing (or called as a cell incubation cassette) includes a first plate 110 and a second plate 120. The second plate 120 is opposite to the first plate 110, and the first plate 110 and the second plate 120 form an incubation cassette 130 and a supply channel 140, wherein the incubation cassette 130 is connected to the supply channel 140 by a microstructure 150.

In one embodiment, the supply channel 140 is a U-shape channel and includes a chamber 145. The chamber 145 is disposed at the bottom of the U-shape channel 140. The incubation cassette 130 is surrounded by the U-shape channel 140, and the microstructure 150 is disposed between the chamber 145 and the incubation cassette 130.

The incubation cassette 130 is a thin culture container formed by the first plate 110 and the second plate 120. The distance between the first plate 110 and the second plate 120 is 0.5 to 3.0 mm, and the distance between two plates is preferably 1.0 mm. The culture device 100 according to one embodiment of the present invention is made of polycarbonate (PC) with high bio-compatibility as material. Two PC plates are attached to each other and form the incubation cassette 130, whose gap between two PC plates is only about 1.0 mm. In another embodiment, the first plate 110 and the second plate 120 can also be made of, for example, a polyester or polyethylene terephthalate (PET) plate or a polytetrafluoroethylene (PTFE) plate. The first plate 110 and the second plate 120 are transparent material. In an embodiment of the present invention, two side plates are made of the flat material with great transparency, so as to be convenient to directly observe the z-axis structure of the incubation cassette and to be highly compatible with the microscopic imaging technology commonly used in the laboratory without the need for additional cutting sections of paraffin-embedded tissues. The side-view of the multi-layered cell samples in the incubation cassette enables instant observation.

In an embodiment of the present invention, the culture device 100 is made of the length of 25 mm and the width of 45 mm of two PC plates, and the two PC plates are micro-machined by a computer numerical control (CNC) machine tool through a digital signal drive milling machine. The conventional microstructure is mostly applied by standard yellow light process. However, the microstructures 150 (whose diameter of each of the pores may be 100-900 microns) in the culture device 100 of this embodiment cannot be produced by the standard yellow light process, so the computer numerical control (CNC) machine tool is used for cutting processing along with the Z axis as the main rotating axis of the tool, for milling work from top to bottom. The first plate 110 is milled downward to a depth of about 1.2 mm to form the spaces of the incubation cassette 130, the supply channel 140 and microstructures 150, after the completion of the preparation, and the first plate 110 and the second plate 120 are glued together by, for example, double-sided adhesive.

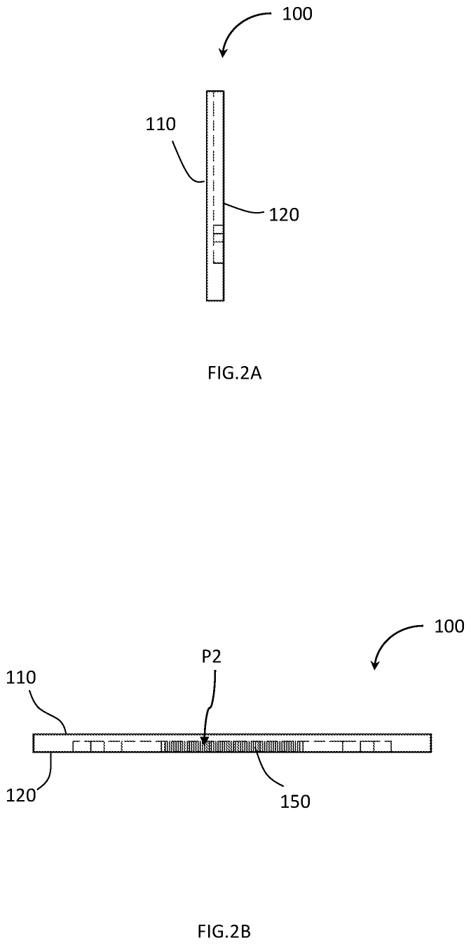

Please refer to FIG. 3, illustrate a front view and a partial enlarged schematic of the culture device 100 for biochemical testing, according to one embodiment of the present invention. The supply channel 140 includes two first openings P1 disposed on the same sides of the first plate 110 and the second plate 120. The incubation cassette 130 includes a second opening P2, and the second opening P2 is disposed on the same sides of the first plate 110 and the second plate 120, where the two first openings P1 are. The supply channel 140 includes a chamber 145 disposed between the two first openings P1, and the chamber 145 is connected to the incubation cassette 130 via the microstructure 150. The microstructure 150 is formed by the staggered arrangement of a plurality of pores 151 and a plurality of sheets 152. The diameter of each of the pores 151 is 100 to 900 μm and is preferably 250 μm. The microstructure 150 is formed by micro-machining technique on the first plate. In this embodiment, a hydrogel L is disposed on a side of the microstructure 150 near the incubation cassette 130. The hydrogel L with permeability near the bottom of the incubation cassette 130 is to increase the bonding efficiency of the cells at the bottom and to block the culture cells C (its size is about tens of microns) falling down from the microstructure 150 (whose each of the pores is about hundreds of microns wide).

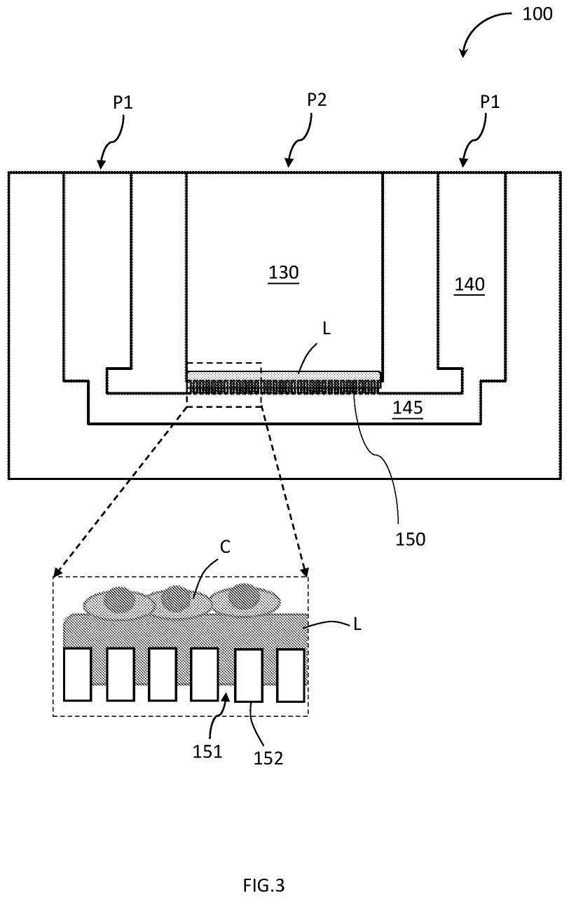

Refer to FIG. 4A, FIG. 4B and FIG. 4C, respectively, show a front view, a top view and a side vide of the physical culture device 100 for biochemical testing, according to one embodiment of the present invention. Due to the need to supply nutrients in a small volume, the special microstructure 150 is processed between the two PC plates to make the nutrient solution supply from the first opening P1 into the supply channel 140 and the nutrient solution chamber 145, for providing the nutrients requirement for long-term cell culture. Furthermore, the design of the second opening P2 at the top of the incubation cassette 130 (or called as an incubation tank) can be directly injected by the micropipette M commonly used in laboratories without the need for additional equipment such as injection pump, and can be combined with the characteristics of the conventional “Transwell” for air-liquid interface (ALI) culture and imitating a highly relevant culture system for respiratory physiology.

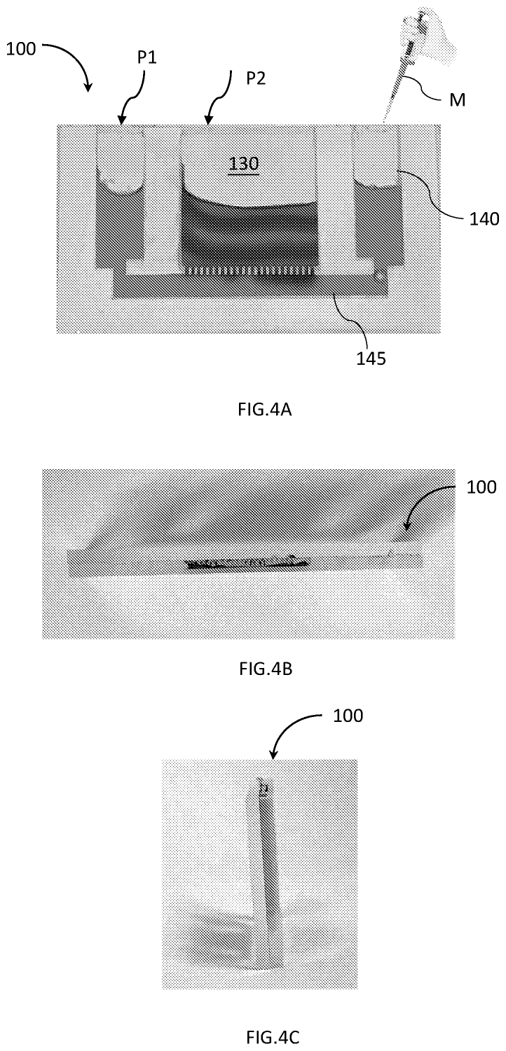

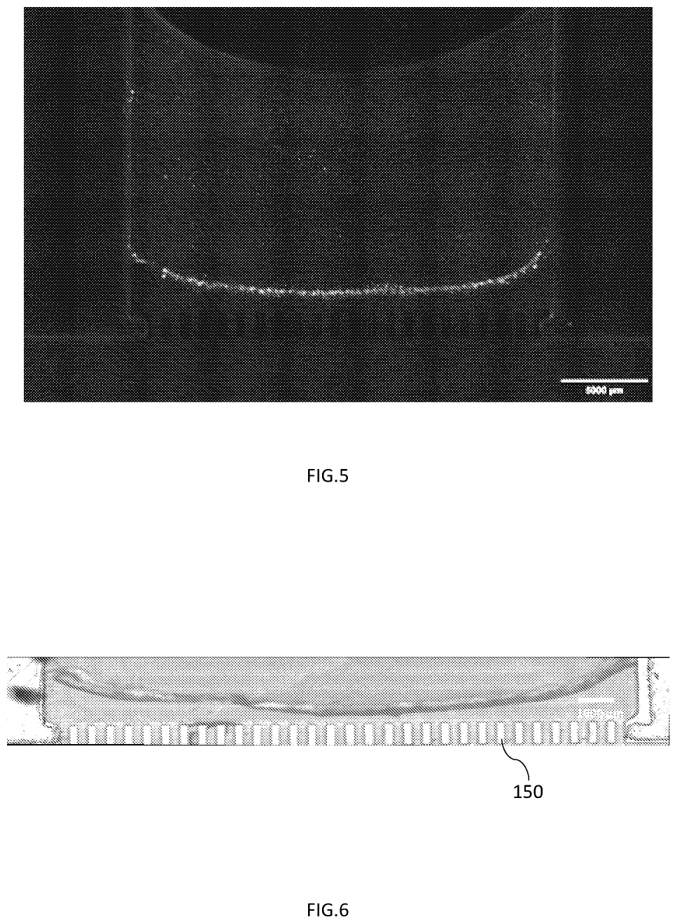

The aim of the culture device for biochemical testing according to one embodiment of the present invention is to provide side views of multi-layered cellular samples for real-time monitoring, which is different from the conventional method that the side view of the sample is obtained by freezing and cutting sections of paraffin-embedded tissues. The culture device 100 for biochemical testing in the embodiment of the present invention can be directly observed the growth status between the multiple layers of cells during the culturing period, as shown in FIG. 5 and FIG. 6. FIG. 5 and FIG. 6, respectively, show a side image (with 5000 μm as the standard scale) and a partial image thereof (with 1000 μm as the standard scale) of the co-culture of lamellar cells in the culture device.

This culture device can be added in a sequential manner to cultivate a layered multicellular tissue structure or co-culture different types of cells. For example, cells added to the culture device at different time points are marked with different fluorescence to achieve the purpose of multi-layered cell culture. In addition, the invasion process of tumors includes the manifestations of malignant phenotypes such as tumor cell proliferation, invasion and migration, and the cells secrete enzymes degrade the ECM (extracellular matrix) and break through the limitation of the basement membrane. This culture device can conveniently provide side images so as to observe the lateral growth of the cells and to monitor the downward invasion of the cells.

The advantages of this culture device for biochemical testing according to one embodiment of the present invention are as following, and the culture device can also meet the following experimental requirements.

1. Provide the real-time side view of the Z axis changes in the cell samples for observation.

2. Easy operate similar to the conventional cell-culture dish, and the special equipment is not required.

3. The design of the opening at the top of the incubation cassette (or called as an incubation tank) imitates the air-liquid interface (ALI) culture and facilitates the culture of three-dimensional layered multicellular structures: by no need of additional support materials between layered structures, and only by adding the cells in a sequential manner.

4. Culture of small volume of the cell samples (in long-term): the space of the incubation cassette is about 50˜200 mm², and there are only need of about 10³˜2*10³ cell samples. By use of the designed nutrient solution chamber (the volume up to 100˜20000) and the supply channel, the culture device can be operated for more than 72 hours without changing the nutrient solution. The nutrient solution is injected and exchanged though the U-shape channel, so the disturbance to the culture cell sample is reduced during the operation. Nevertheless, in the conventional small-area-sized conventional cell-culture dish (eg, 96 wells), there are requirement of at least about 10⁴ cell samples and the storage volume of the nutrient solution is only 100-200 μl, which is hardly conducive to long-term culture.

What is claimed is:

1. A culture device for biochemical testing, comprising:
a first plate; and
a second plate, opposite to the first plate, and the first plate and the second plate form an incubation cassette and a supply channel,
wherein the incubation cassette is connected to the supply channel by a micro structure.

2. The culture device of claim 1, wherein the incubation cassette is a thin culture container formed by the first plate and the second plate, and the distance between the first plate and the second plate is 0.5 to 3.0 mm.

3. The culture device of claim 1, wherein the supply channel is a U-shape channel, and comprises a chamber, the chamber is disposed at the bottom of the U-shape channel, the incubation cassette is within the U-shape channel, and the microstructure is disposed between the chamber and the incubation cassette.

4. The culture device of claim 1, wherein the supply channel comprises two first openings disposed on the same sides of the first plate and the second plate.

5. The culture device of claim 4, wherein the incubation cassette comprises a second opening, and the second opening is disposed on the same sides of the first plate and the second plate, where the two first openings are.

6. The culture device of claim 4, wherein the supply channel comprises a chamber disposed between the two first openings, and the chamber is connected to the incubation cassette via the microstructure.

7. The culture device of claim 1, wherein a hydrogel is disposed on a side of the microstructure near the incubation cassette.

8. The culture device of claim 1, wherein the microstructure is formed by the staggered arrangement of a plurality of pores and a plurality of sheets.

9. The culture device of claim 8, wherein the diameter of each of the pores is 100 to 900 μm.

10. The culture device of claim 1, wherein the microstructure is formed by micro-machining technique.

11. The culture device of claim 1, wherein the first plate and the second plate are transparent material, and comprise a PC plate, a PET plate or a PTFE plate.
